(19) United States
(12) Patent Application Publication
(10) Pub. No.: US 2015/0313636 A1
(43) Pub. Date: Nov. 5, 2015

(54) Device and Methods to Reduce Vaginal Tearing During Delivery

(71) Applicants: Daniel Z. SHEN, Stanford, CA (US); Kabir James YAMANA, Cambridge, MA (US)

(72) Inventors: Daniel Zhiling SHEN, Stanford, CA (US); Kabir James YAMANA, Cambridge, MA (US)

(21) Appl. No.: 14/717,726

(22) PCT Filed: Nov. 20, 2013
(86) PCT No.: PCT/US2013/071092

§ 371 Date: May 20, 2015

Related U.S. Application Data
(60) Provisional application No. 61/728,756, filed on Nov. 20, 2012.

(51) Int. Cl. A61B 17/42; A61F 5/00

(52) U.S. Cl. CPC A61B 17/42; A61F 5/0093; A61B 2017/00951

(57) ABSTRACT

A perineal protection device for use during childbirth comprises one or more pads configured to be placed on the perineum and having a varying level of resistance to stretch along the anterior-posterior axis between the vaginal opening and the anus. The device further includes attachment means in the form of adhesives, hooks, sutures, or other suitable means, for adhering the pad to the skin during childbirth, with the result that stress on the perineum is reduced during childbirth, thus reducing tearing during childbirth. The resistance to stretch is relatively lower near the vaginal opening, and relatively greater near the anus. The increase can be linear or nonlinear. The pad can be formed monolithically or in multiple portions. The resistance to stretch can be varied by varying the modulus of elasticity, varying cross-sectional area, or other suitable mechanical or materials-based techniques.

### RELATED APPLICATIONS

This application is a Section 371 conversion of PCT Application No. PCT/US2013/71092, filed on Nov. 20, 2013, which in turn is a conversion of Provisional Patent Application Ser. No. 61/728,756, filed Nov. 20, 2012. The present application claims the benefit of priority of each of the foregoing applications, all of which are incorporated herein for all purposes.

### FIELD OF THE INVENTION

The present invention relates generally to medical devices and methods for assisting during delivery, and more particularly relates to medical devices affixed to the pregnant mother during delivery, and methods therefor.

### BACKGROUND OF THE INVENTION

Perineal tearing has long been an issue that pregnant mothers face during childbirth. Delivery can lead to overstretching of the vagina, causing tears in the perineal tissue between the vagina and the rectum, as the vagina of the mother stretches to accommodate the passage of the baby's body through the birth canal. In some instances, an episiotomy is performed as a prophylactic measure, to prevent uncontrolled tearing between the vagina and the anus.

Perineal tears or lacerations are typically classified to indicate the severity of the trauma to the perineum, as follows:

First degree tear: laceration is limited to the fourchette and superficial perineal skin or vaginal mucosa.

Second degree tear: laceration extends beyond fourchette, perineal skin and vaginal mucosa to perineal muscles and fascia, but not the anal sphincter.

Third degree tear: fourchette, perineal skin, vaginal mucosa, muscles, and anal sphincter are torn. Third degree tears are further characterized as “3a”, “3b”, and “3c”, where 3a is characterized by partial tear of the external anal sphincter involving less than 50% thickness, 3b is characterized by greater than 50% tear of the external anal sphincter, and 3c is characterized in that the internal sphincter is torn.

Fourth degree tear: fourchette, perineal skin, vaginal mucosa, muscles, sphincter, and rectal mucosa are torn.

Such tearing can cause significant post-delivery complications for the mother. As a result, there has long been a need to reduce the rate and nature of perineal tears without hindering or preventing the vaginal stretching needed to facilitate delivery.

### SUMMARY OF THE INVENTION

The present invention comprises a device that provides support to the tissues comprising and bordering the line between the bottom of the vagina and the anus, and methods relating thereto. A component of the support, which can be a large component in some embodiments, is oriented in a direction perpendicular to the line between the vagina and the anus.

In an embodiment, the support is not uniform, with comparatively greater support being provided near the anus, and less near the vagina. In an embodiment, the increase in support can be linear. The support near the vagina can, in an embodiment, be smaller to allow for some tearing near the vagina, while providing greater support near the anus with the objective of making tears near the anus unlikely.

In an embodiment, the support can comprise a force that acts on the skin and, directly or indirectly, the underlying tissues, or can be elastic, to generate a force that resists stretching and tearing of the tissue. The desired support can be provided by any of a group of methods including a variable stiffness material or plurality of materials, a fixed or variable spring constant, an elastic members with variable spring constants, or by a material which applies variable force curves to the skin as the device is stretched.

The device can be secured to the skin bordering the ano-vaginal line using methods taken from a group comprising: adhesives suited for use on skin, high friction materials, hooks, clips, pins, protrusions, sutures, staples, straps, external members, or any other method that suitably increases the friction or sheer force of the device against the skin.

These and other aspects of the invention can be appreciated from the following Detailed Description of the Invention, taken together with the appended Figures, described below.

### THE FIGURES

FIGS. 1-8 show a generalized embodiment and a plurality of alternative embodiments of a perineal support device in accordance with the present invention.

### DETAILED DESCRIPTION OF THE INVENTION

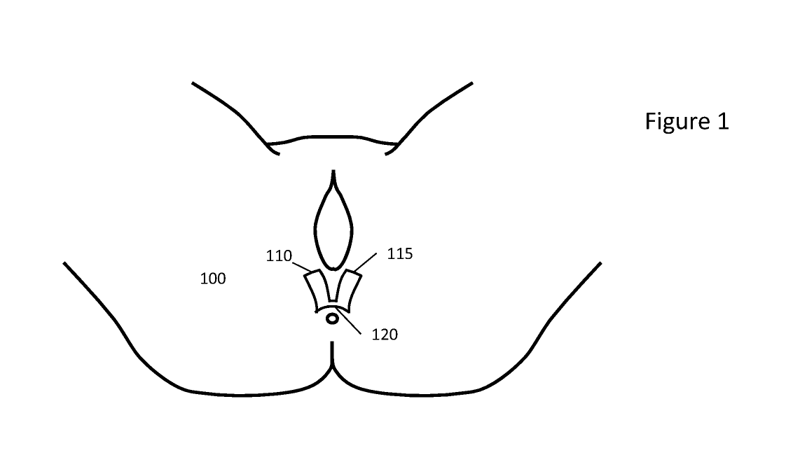

Referring first to FIG. 1, a generalized embodiment of a perineal protection device 100 can be appreciated. The device 100 comprises two halves 110-115, joined near the anus. In an embodiment, each half can be relatively rigid on its own, and joined by a hinge 120 or other connector. The hinge can be a multi-part hinge with a pin connecting the two halves. In such an arrangement, the two halves are pulled toward the center line by a spring component in order to limit tearing. The spring component can be provided by any suitable means, for example a torsional spring at the pin. The hinge can alternatively be monolithic, and integrated with the device halves, with the monolithic hinge being a thinner part of the same material as the two halves, and thus more flexible. This results in the monolithic hinge acting both as a flexible member to allow the two halves to move apart as needed, and also to provide a spring force holding the two halves together to reduce tearing of the tissues. In an embodiment, the two halves are both relatively rigid such that the majority of the flexing occurs at the hinge. The amount of skin separation (stretching and tearing) in such an arrangement is generally proportional to the distance from the hinge axis. In a different embodiment, the two halves can be flexible, and, in a still further embodiment, can be more flexible as they get closer to the vagina, to allow for more stretching at the vagina while providing increasing stiffness as the distance from the anus decreases.

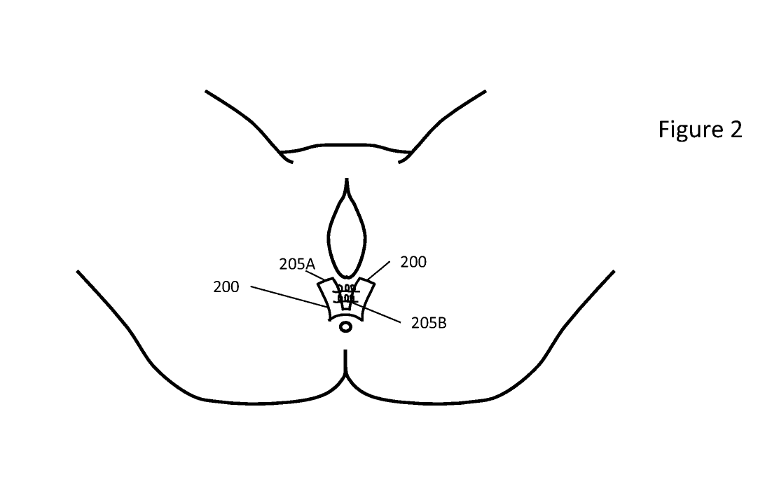

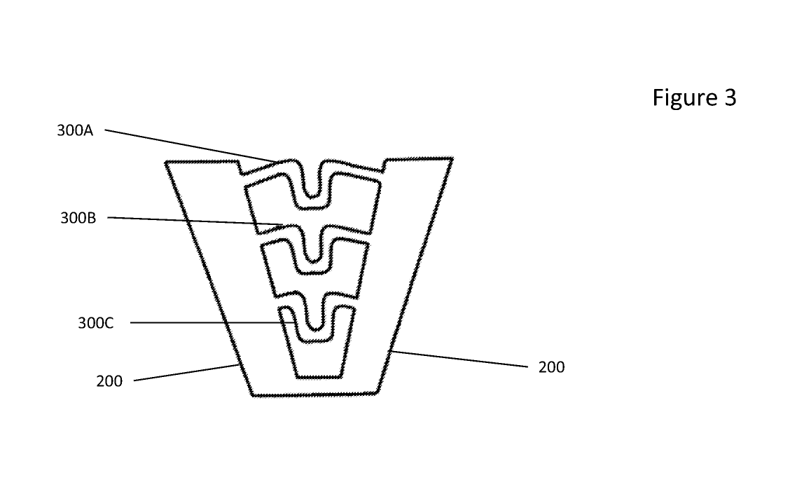

Referring next to FIG. 2, the embodiment shown therein illustrates an alternative method of achieving variable elasticity, with stiffness increasing along the line toward the anus. In particular, halves 200 are connected by one or more tensile or elastic members 205A-B. The members 205 can be of varying stiffness, with the member having the greatest stiffness being proximate to the anus, and the member having the least stiffness being proximate to the vagina. The members 205A-B can be metal springs, rubber bands, elastic fibers, flexible plastic, textiles, or monolithic springs formed integrally with the two halves 200. Each of the members flexes to allow the two halves to separate. The members can be “U” shaped as shown in FIG. 3 for members 300A-C, but can also be any other convenient shape known to those skilled in the art; for example, each elastic member can have a different thickness to provide varying resistance to the separation of the two halves.

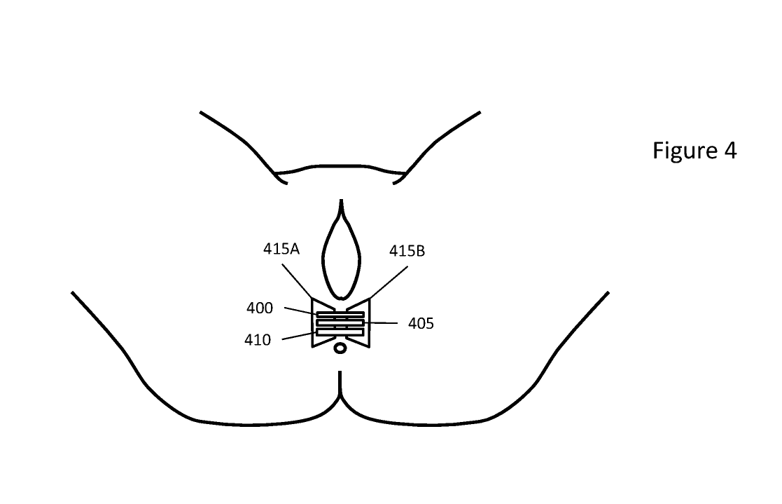

Referring next to FIG. 4, the use of elastic members having variable elasticity can be better appreciated. In particular, elastomers 400, 405 and 410 are each connected to halves 415A-B, with elastomer 400, nearest the vagina, having the lowest modulus of elasticity or cross sectional area, and elastomer 410, nearest the anus, having the highest.

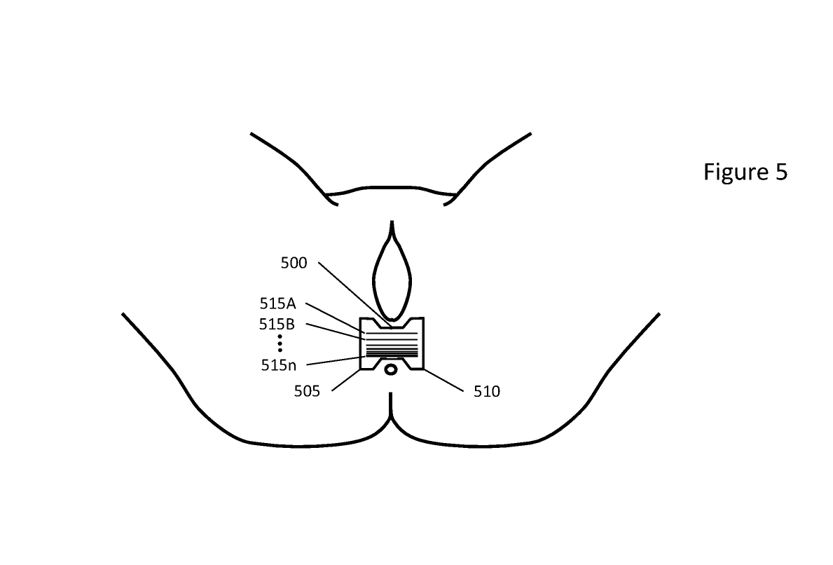

Referring next to FIG. 5, a monolithic embodiment of the invention is illustrated. A stretchable center patch 500 is formed integrally with two halves 505 and 510. The stretchable center patch 500 comprises a plurality of stretchable members 515A-n, with the rigidity of the center patch increasing as the distance to the anus decreases. The rigidity of the center patch can be increased either by increasing the rigidity of the individual members, or by increasing the density (i.e., the number per area) of stretchable members as the distance to the anus decreases.

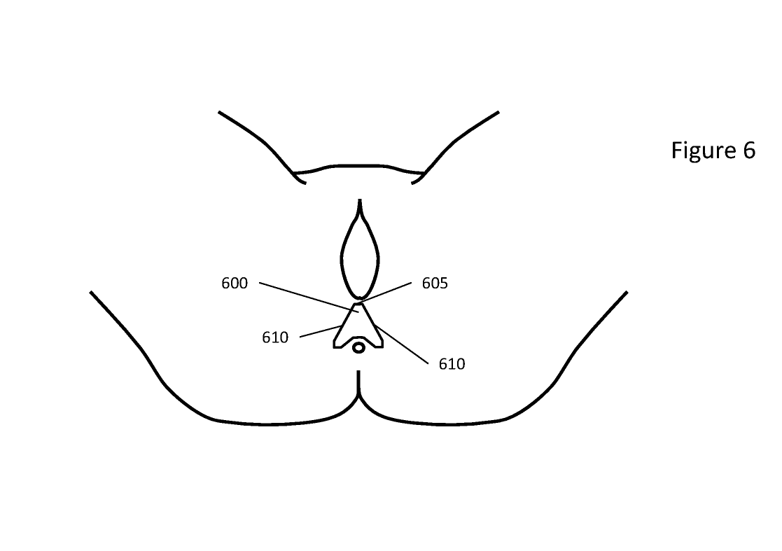

Referring next to FIG. 6, a still further alternative approach can be better appreciated. In some births, tearing is unavoidable. In such circumstances, the objective is to limit the tearing and to prevent the tear from propagating along the centerline from the vagina to the anus. The embodiment shown in FIG. 6 achieves this by directing the tear along the edge of a triangular shaped element 600, whereby the tear is directed away from the anus. In the embodiment shown, the triangular element 600 is applied to the perineal area with the point of the triangle 605 directed toward the posterior of the vagina. The element 600 is applied by any suitable method, including adhesives and the other methods mentioned hereinabove. By having the narrow, pointed portion nearest the bottom of the vagina, stretching of the vagina is allowed. Should a tear occur, the perineal tissue is supported by the device 600, which directs the tear along an edge 610 of the device rather than along the centerline, since the key stresses on the perineal tissue form at the edge of the device 600.

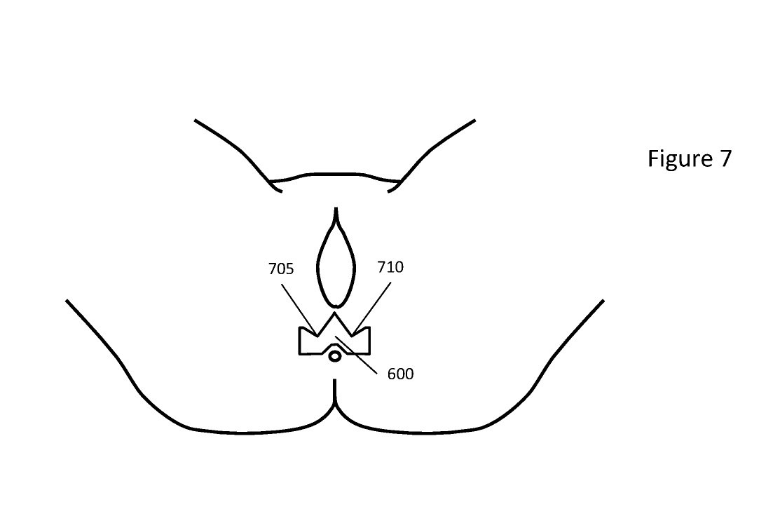

In some instances, it may be desirable not merely to direct the tear away from the anus, but also to support the skin sufficiently that the redirected tear is stopped. An embodiment for achieving this result is shown in FIG. 7, where the substantially triangular device 600 of FIG. 6 now includes a pair of V-shaped notches 705-710, one on either side of the centerline.

It will be appreciated that a caregiver can apply pressure to help hold the device of the present invention in place. The present invention, at the least, helps regulate the amount of inward/medial pressure exerted on the skin and underlying structures. Many embodiments can also have tactile or other features on the surface to help guide the caregiver's fingers to the optimal points to apply a force normal to the surface of the skin.

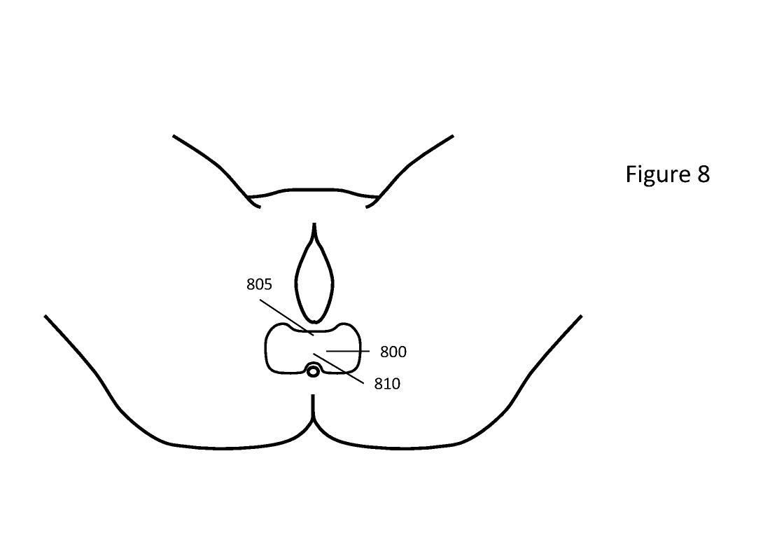

In another embodiment, shown in FIG. 8, the device can comprise primarily a single pad of elastomeric material, indicated at 800. In an embodiment, the pad can vary in thickness over the area of the patch. In areas where the skin will be allowed to stretch more, such as near the vagina, the pad can be configured to be thinner, as shown at 805. In areas where more support is needed to prevent excessive stretching, such as near the anus, the pad can be configured to be thicker, as shown at 810. The pad may aid the user in holding onto or connecting the skin and tissue with the pad. In one embodiment, an adhesive can be used to provide adhesion to the skin and this adhesive can be flexible to allow stretching of the underlying skin while still maintaining adhesion. In other embodiments, hooks, needles, high friction surfaces, and other suitable means can be used to help hold the pad onto the skin and reduce sliding of the skin and tissue under the pad. Note that for those embodiments which use an elastomeric material can also use other materials for the pad that stretch and provide more resistance when stretched. In another embodiment, the pad material may also vary to achieve varying resistance when stretched.

In one embodiment, the desired varying resistance under stretch is determined via an analysis of the stretching of the perineal skin during birth that yields a model of typical elongation of the skin. This model of elongation can be used to optimize the thickness, shape, density and/or material composition of the pad in different areas of the pad.

In one embodiment, the pad can be designed to provide a constant support force in reaction to the typical stretching, and areas that typically do not stretch much, such as near the anus, will be supported more when they are stretched beyond the typical amount. In an embodiment, the pad can be made of silicone, though any suitable elastomeric material can be used. In another embodiment the pad can contain other materials. These materials, which can be chosen from a group comprising filaments, threads, fabrics, textiles, wires, different elastomeric materials, other plastics, and springs, can affect the support forces provided by areas of the pad and help better control or vary these forces. For example, threads may be added to the pad to increase the support around the anus. These threads can be selectively aligned within the pad to customize the support in different directions. Different pads can be made for different perineal shapes and sizes. There may be a number of common sizes available. Custom pads can be made for a given user as well. Although a single pad design is mentioned above, other embodiments can have multiple pads connected together by elastic or non-elastic members.

In an embodiment, any of the above designs can be modified to provide the ability for a doctor or midwife to adjust the support provided by the device during childbirth. Such an adjustment method can be achieved by providing an elastic layer that can be attached to the device at some point, and can also be peeled off, either to cause the device to provide more support, or to allow the device to become more flexible. The device itself can be designed to be easily peeled from the skin.

From the foregoing, it can be appreciated that a new and novel perineal protection device has been disclosed. Having fully described several embodiments in detail, it will be apparent to those skilled in the art, given the teachings herein, that numerous alternatives and equivalents exist which are within the scope of the invention. Therefore, the foregoing description is not to be interpreted as limiting, and the scope of the invention is to be limited only by the appended claims.

We claim:

1. A perineal protection device for use during childbirth comprising
first and second portions configured to be positioned substantially symmetrically along an imaginary line connecting the vaginal opening and the anus during delivery,
attachment means for affixing each portion to the skin, and
at least one displaceable member for connecting the first and second portions and for permitting the first and second portions to move away from one another during delivery.

2. A perineal protection device for use during childbirth comprising
at least one pad configured to be placed on the perineum and having a varying level of resistance to stretch along the anterior-posterior axis between the vaginal opening and the anus, and
attachment means for adhering the pad to the skin during childbirth to reduce the stress on the perineum and thus reduce tearing during childbirth.

3. The perineal protection device of claim 2 wherein the pad comprises a plurality of pads configured to vary the resistance to stretch, with less resistance at the area nearest the vaginal opening, and greater resistance to stretch nearest the anus.

4. The perineal protection device of claim 1 wherein the displaceable member is one of a spring or an elastic member.

5. The perineal protection device of claim 1 wherein the displaceable member is formed monolithically with the first and second portions and provides less resistance to stretch than the first and second portions.

6. The perineal protection device of claim 2 wherein the pad is formed monolithically and has the relatively lower modulus of elasticity nearer the vaginal opening and the relatively greater modulus of elasticity nearer the anus.

7. The perineal protection device of claim 6 wherein the increase in modulus of elasticity is substantially linear.

8. The perineal protection device of claim 2 wherein the pad has a smaller cross-sectional area nearer the vaginal opening and a greater cross-sectional area nearer the anus.
